(19) United States
(12) Patent Application Publication
(10) Pub. No.: US 2026/0354889 A1
(43) Pub. Date: Nov. 12, 2026

(54) SURGICAL INSTRUMENTS, SYSTEMS, AND METHODS INCORPORATING ULTRASONIC AND ELECTROSURGICAL FUNCTIONALITY

(71) Applicant: Covidien LP, Mansfield, MA (US)

(72) Inventors: Kristen P. BRADLEY, Westminster, CO (US); David J. VAN TOL, Boulder, CO (US); Michael B. LYONS, Boulder, CO (US)

(73) Assignee: Covidien LP, Mansfield, MA (US)

(21) Appl. No.: 19/102,084

(22) PCT Filed: Aug. 1, 2023
(86) PCT No.: PCT/IB2023/057788

§ 371 Date: Feb. 7, 2025

Related U.S. Application Data
(60) Provisional application No. 63/395,942, filed on Aug. 8, 2022.

(51) Int. Cl. A61B 18/14; A61B 17/32; A61B 18/00

(52) U.S. Cl. CPC A61B 18/1447; A61B 2017/320074; A61B 2017/320095; A61B 2018/0019; A61B 2018/0063; A61B 2018/00755; A61B 2018/00767; A61B 2018/00994

(57) ABSTRACT

A surgical system includes an electrosurgical system including first and second electrodes configured to conduct electrosurgical energy therebetween and through tissue to treat tissue and an ultrasonic system that is configured to ultrasonically vibrate an ultrasonic blade to treat tissue in contact therewith. In a first mode of operation, the electrosurgical system is configured to conduct the electrosurgical energy according to a first electrosurgical algorithm and the ultrasonic system is configured to ultrasonically vibrate the ultrasonic blade according to a first ultrasonic algorithm. In a second mode of operation, the electrosurgical system is configured to conduct the electrosurgical energy according to a second electrosurgical algorithm and the ultrasonic system is configured to ultrasonically vibrate the ultrasonic blade according to a second ultrasonic algorithm. The first and second electrosurgical algorithms are different from one another and/or the first and second ultrasonic algorithms are different from one another.

### FIELD

The present disclosure relates to energy based surgical instruments and, more particularly, to surgical instruments and systems incorporating ultrasonic and electrosurgical functionality to facilitate energy-based tissue treatment.

### BACKGROUND

Ultrasonic surgical instruments and systems utilize ultrasonic energy, i.e., ultrasonic vibrations, to treat tissue. More specifically, ultrasonic surgical instruments and systems utilize mechanical vibration energy transmitted at ultrasonic frequencies to treat tissue. An ultrasonic surgical device may include, for example, an ultrasonic blade and a clamp mechanism to enable clamping of tissue against the blade. Ultrasonic energy transmitted to the blade causes the blade to vibrate at very high frequencies, which allows for heating tissue to treat tissue clamped against or otherwise in contact with the blade.

Electrosurgical instruments and systems conduct Radio Frequency (RF) energy through tissue to treat tissue. An electrosurgical instrument or system may be configured to conduct bipolar RF energy between oppositely charged electrodes and through tissue, e.g., tissue clamped between the electrodes or otherwise in contact therewith, to treat tissue. Alternatively or additionally, an electrosurgical instrument or system may be configured to deliver monopolar RF energy from an active electrode to tissue in contact with the electrode, with the energy returning via a local or remote return electrode device to complete the circuit.

### SUMMARY

As used herein, the term “distal” refers to the portion that is described which is farther from an operator (whether a human surgeon or a surgical robot), while the term “proximal” refers to the portion that is being described which is closer to the operator. Terms including “generally,” “about,” “substantially,” and the like, as utilized herein, are meant to encompass variations, e.g., manufacturing tolerances, material tolerances, use and environmental tolerances, design variations, measurement variations, and/or other variations, up to and including plus or minus 10 percent. Further, any or all of the aspects described herein, to the extent consistent, may be used in conjunction with any or all of the other aspects described herein.

Provided in accordance with aspects of the present disclosure is a surgical system including an electrosurgical system and an ultrasonic system. The electrosurgical system includes first and second electrodes configured to conduct electrosurgical energy therebetween and through tissue to treat tissue. The ultrasonic system includes an ultrasonic blade and is configured to ultrasonically vibrate the ultrasonic blade to treat tissue in contact with the ultrasonic blade. In a first mode of operation, the electrosurgical system is configured to conduct the electrosurgical energy according to a first electrosurgical algorithm and the ultrasonic system is configured to ultrasonically vibrate the ultrasonic blade according to a first ultrasonic algorithm. In a second mode of operation, the electrosurgical system is configured to conduct the electrosurgical energy according to a second electrosurgical algorithm and the ultrasonic system is configured to ultrasonically vibrate the ultrasonic blade according to a second ultrasonic algorithm. The first and second electrosurgical algorithms are different from one another and/or the first and second ultrasonic algorithms are different from one another.

In an aspect of the present disclosure, the surgical system further includes a jaw member movable relative to the ultrasonic blade for clamping tissue between the jaw member and the ultrasonic blade. In such aspects, the jaw member includes one of the first or second electrodes and the ultrasonic blade includes the other of the first or second electrodes. Alternatively, the jaw member may include both of the first and second electrodes.

In another aspect of the present disclosure, in at least one of the first or second modes of operation, the electrosurgical energy is conducted and the ultrasonic blade is ultrasonically vibrated simultaneously.

In still another aspect of the present disclosure, the first mode of operation is a tissue sealing mode of operation and the second mode of operation is a tissue transection mode of operation.

In yet another aspect of the present disclosure, the first electrosurgical algorithm includes a variable function and the second electrosurgical algorithm includes a constant function. In aspects, the variable function includes a polynomial function.

In still yet another aspect of the present disclosure, the first electrosurgical algorithm includes controlling power based on impedance and the second electrosurgical algorithm includes voltage control, e.g., based on impedance.

In another aspect of the present disclosure, the first electrosurgical algorithm includes a plurality of different stages and the second electrosurgical algorithm includes a single stage.

In yet another aspect of the present disclosure, the first ultrasonic algorithm includes a constant function at a first power level and the second ultrasonic algorithm includes a constant function at a second, different power level.

In still another aspect of the present disclosure, the first ultrasonic algorithm includes a variable function and the second ultrasonic algorithm includes a constant function.

A surgical method provided in accordance with aspects of the present disclosure includes receiving an input to operate in a first mode of operation or a second mode of operation. In response to receiving the input to operate in the first mode of operation, the method includes conducting electrosurgical energy between first and second electrodes and through tissue according to a first electrosurgical algorithm and ultrasonically vibrating an ultrasonic blade in contact with tissue according to a first ultrasonic algorithm. In response to receiving the input to operate in the second mode of operation, the method includes conducting electrosurgical energy between the first and second electrodes and through tissue according to a second electrosurgical algorithm and ultrasonically vibrating the ultrasonic blade in contact with tissue according to a second ultrasonic algorithm. The first and second electrosurgical algorithms are different from one another and/or the first and second ultrasonic algorithms are different from one another.

In an aspect of the present disclosure, in the first and/or second modes of operation, the conducting electrosurgical energy and the ultrasonically vibrating are performed simultaneously.

In another aspect of the present disclosure, the first mode of operation is a tissue sealing mode of operation and the second mode of operation is a tissue transection mode of operation.

In yet another aspect of the present disclosure, the conducting electrosurgical energy according to the first electrosurgical algorithm includes implementing a variable function and the conducting electrosurgical energy according to the second electrosurgical algorithm includes implementing a constant function. In such aspects, the variable function may include a polynomial function.

In still another aspect of the present disclosure, the conducting electrosurgical energy according to the first electrosurgical algorithm includes controlling power based on impedance and the conducting electrosurgical energy according to the second electrosurgical algorithm includes implementing voltage control, e.g., based on impedance.

In still yet another aspect of the present disclosure, the conducting electrosurgical energy according to the first electrosurgical algorithm includes implementing a plurality of electrosurgical energy delivery stages and the conducting electrosurgical energy according to the second electrosurgical algorithm includes implementing a single electrosurgical energy delivery stage.

In an aspect of the present disclosure, the ultrasonically vibrating according to the first ultrasonic algorithm includes implementing a constant function at a first power level and the ultrasonically vibrating according to the second ultrasonic algorithm includes implementing a constant function at a second, different power level.

In another aspect of the present disclosure, the ultrasonically vibrating according to the first ultrasonic algorithm includes implementing a variable function and the ultrasonically vibrating according to the second ultrasonic algorithm includes implementing a constant function.

### BRIEF DESCRIPTION OF THE DRAWINGS

The above and other aspects and features of the present disclosure will become more apparent in light of the following detailed description when taken in conjunction with the accompanying drawings wherein like reference numerals identify similar or identical elements.

FIG. 1 is a side view of a surgical system provided in accordance with the present disclosure including a surgical instrument, a surgical generator, and a return electrode device;

FIG. 2 is perspective view of another surgical system provided in accordance with the present disclosure including a surgical instrument incorporating an ultrasonic generator, electrosurgical generator, and power source therein;

FIG. 3 is a schematic illustration of a robotic surgical system provided in accordance with the present disclosure;

FIG. 4 is a longitudinal, cross-sectional view of a distal end portion of the surgical instrument of FIG. 1;

FIG. 5 is a transverse, cross-sectional view of the end effector assembly of the surgical instrument of FIG. 1;

FIG. 6 is a transverse, cross-sectional view of another configuration of the end effector assembly of the surgical instrument of FIG. 1;

FIG. 7 is a simplified block diagram of an ultrasonic control system configured for use with the surgical systems of FIGS. 1-3 or any other suitable surgical system;

FIG. 8 is a simplified block diagram of an electrosurgical control system configured for use with the surgical systems of FIGS. 1-3 or any other suitable surgical system;

FIG. 9 is a flow diagram of a tissue treating method provided in accordance with aspects of the present disclosure;

FIGS. 10 and 11 are flow diagrams of tissue sealing methods provided in accordance with aspects of the present disclosure; and

FIGS. 12A and 12B are detailed flow diagrams of portions of the tissue sealing method of FIG. 11.

### DETAILED DESCRIPTION

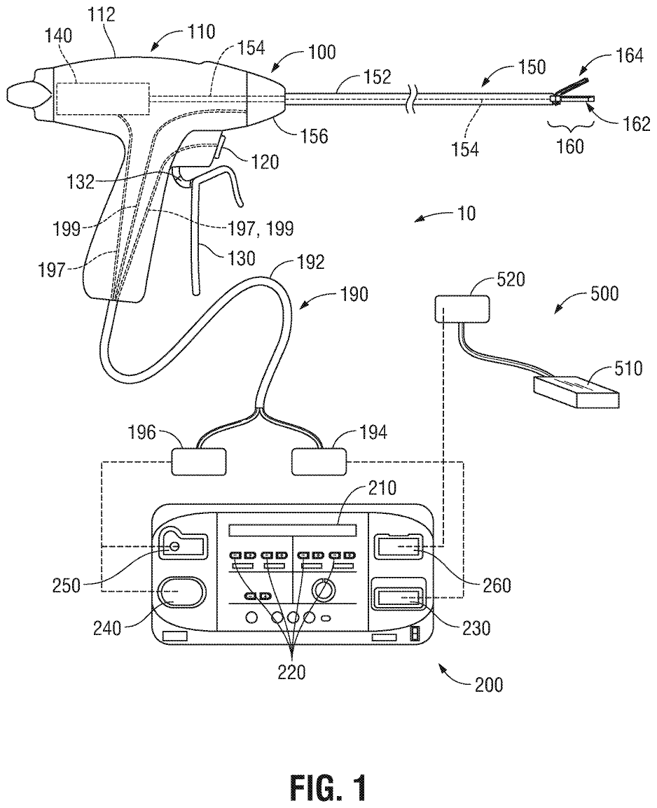

Referring to FIG. 1, a surgical system provided in accordance with aspects of the present disclosure is shown generally identified by reference numeral 10 and includes a surgical instrument 100, a surgical generator 200, and, in some aspects, a return electrode device 500, e.g., including a return pad 510. Surgical instrument 100 includes a handle assembly 110, an elongated assembly 150 extending distally from handle assembly 110, an end effector assembly 160 disposed at a distal end of elongated assembly 150, and a cable assembly 190 operably coupled with handle assembly 110 and extending therefrom for connection to surgical generator 200.

Surgical generator 200 includes a display 210, a plurality user interface features 220, e.g., buttons, touch screens, switches, etc., an ultrasonic plug port 230, a bipolar electrosurgical plug port 240, and active and return monopolar electrosurgical plug ports 250, 260, respectively. As an alternative to plural dedicated ports 230-260, one or more common ports (not shown) may be configured to act as any two or more of ports 230-260.

Surgical instrument 100 is configured to supply electrosurgical, e.g., Radio Frequency (RF), energy to tissue to treat tissue, e.g., in a monopolar configuration and/or a bipolar configuration, and/or to supply ultrasonic energy to tissue to treat tissue. Surgical generator 200 is configured to produce ultrasonic drive signals for output through ultrasonic plug port 230 to surgical instrument 100 to activate surgical instrument 100 to supply ultrasonic energy and to provide electrosurgical energy, e.g., RF bipolar energy for output through bipolar electrosurgical plug port 240 and, in aspects, RF monopolar energy for output through active monopolar electrosurgical port 250 to surgical instrument 100 to activate surgical instrument 100 to supply electrosurgical energy. Plug 520 of return electrode device 500 is configured to connect to return monopolar electrosurgical plug port 260 to return monopolar electrosurgical energy from surgical instrument 100 during monopolar electrosurgical use.

Continuing with reference to FIG. 1, handle assembly 110 includes a housing 112, an activation button 120, and a clamp lever 130. Housing 112 is configured to support an ultrasonic transducer 140. Ultrasonic transducer 140 may be permanently engaged within housing 112 or removable therefrom. Ultrasonic transducer 140 includes a piezoelectric stack or other suitable ultrasonic transducer components electrically coupled to surgical generator 200, e.g., via one or more of first electrical lead wires 197, to enable communication of ultrasonic drive signals to ultrasonic transducer 140 to drive ultrasonic transducer 140 to produce ultrasonic vibration energy that is transmitted along a waveguide 154 of elongated assembly 150 to blade 162 of end effector assembly 160 of elongated assembly 150, as detailed below. Feedback and/or control signals may likewise be communicated between ultrasonic transducer 140 and surgical generator 200. Ultrasonic transducer 140, more specifically, may include a stack of piezoelectric elements secured, under pre-compression between proximal and distal end masses or a proximal end mass and an ultrasonic horn with first and second electrodes electrically coupled between piezoelectric elements of the stack of piezoelectric elements to enable energization thereof to produce ultrasonic energy. However, other suitable ultrasonic transducer configurations, including plural transducers and/or non-longitudinal, e.g., torsional, transducers are also contemplated.

An activation button 120 is disposed on housing 112 and coupled to or between ultrasonic transducer 140 and/or surgical generator 200, e.g., via one or more of first electrical lead wires 197, to enable activation of ultrasonic transducer 140 in response to depression of activation button 120. In some configurations, activation button 120 may include an ON/OFF switch. In other configurations, activation button 120 may include multiple actuation switches to enable activation from an OFF state to different states corresponding to different activation modes, e.g., a first state corresponding to a first activation mode (such as a tissue sealing mode) and a second state corresponding to a second activation mode (such as a tissue transection mode). In still other configurations, separate activation buttons may be provided, e.g., a first actuation button for activating a first activation mode and a second activation button for activating a second activation mode. Additional activation buttons, sliders, wheels, etc. are also contemplated to enable control of various different activation modes from housing 112.

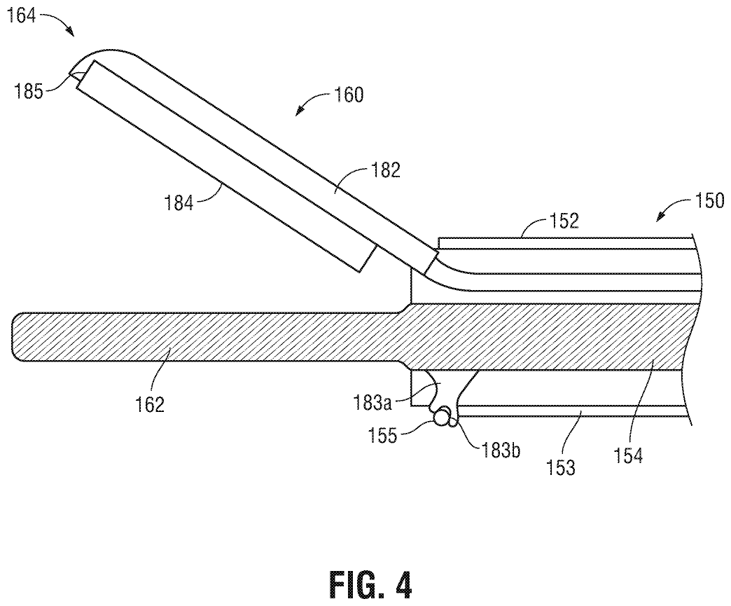

Elongated assembly 150 of surgical instrument 100 includes an outer drive sleeve 152, an inner support sleeve 153 (FIG. 4) disposed within outer drive sleeve 152, a waveguide 154 extending through inner support sleeve 153 (FIG. 4), a drive assembly (not shown), a rotation knob 156, and an end effector assembly 160 including a blade 162 and a jaw member 164. Rotation knob 156 is rotatable in either direction to rotate elongated assembly 150 in either direction relative to handle assembly 110. The drive assembly operably couples a proximal portion of outer drive sleeve 152 to clamp lever 130 of handle assembly 110. A distal portion of outer drive sleeve 152 is operably coupled to jaw member 164 and a distal end of inner support sleeve 153 (FIG. 4) pivotably supports jaw member 164. As such, clamp lever 130 is selectively actuatable, e.g., between an un-actuated position and a fully actuated position, to thereby move outer drive sleeve 152 about inner support sleeve 153 (FIG. 4) to pivot jaw member 164 relative to blade 162 of end effector assembly 160 from an open position towards a closed position for clamping tissue between jaw member 164 and blade 162. The configuration of outer and inner sleeves 152, 153 (FIG. 4) may be reversed, e.g., wherein outer sleeve 152 is the support sleeve and inner sleeve 153 (FIG. 4) is the drive sleeve. Other suitable drive structures as opposed to a sleeve are also contemplated such as, for example, drive rods, drive cables, drive screws, etc.

Referring still to FIG. 1, the drive assembly may be tuned to provide a jaw clamping force, or jaw clamping force within a jaw clamping force range, to tissue clamped between jaw member 164 and blade 162, such as described in U.S. Patent Application Pub. No. 2022/0117622, published on Apr. 21, 2022, the entire contents of which are hereby incorporated herein by reference. Alternatively, the drive assembly may include a force limiting feature, e.g., a spring, whereby the clamping force applied to tissue clamped between jaw member 164 and blade 162 is limited to a particular jaw clamping force or a jaw clamping force within a jaw clamping force range, such as described in U.S. Pat. No. 10,368,898, issued on Aug. 6, 2019, the entire contents of which are hereby incorporated herein by reference.

Waveguide 154, as noted above, extends from handle assembly 110 through inner sleeve 153 (FIG. 4). Waveguide 154 includes blade 162 disposed at a distal end thereof. Blade 162 may be integrally formed with waveguide 154, separately formed and subsequently attached (permanently or removably) to waveguide 154, or otherwise operably coupled with waveguide 154. Waveguide 154 and/or blade 162 may be formed from titanium, a titanium alloy, or other suitable electrically conductive material(s), although non-conductive materials are also contemplated. Waveguide 154 includes a proximal connector (not shown), e.g., a threaded male connector, configured for engagement, e.g., threaded engagement within a threaded female receiver, of ultrasonic transducer 140 such that ultrasonic motion produced by ultrasonic transducer 140 is transmitted along waveguide 154 to blade 162 for treating tissue clamped between blade 162 and jaw member 164 or positioned adjacent to blade 162.

Cable assembly 190 of surgical instrument 100 includes a cable 192, an ultrasonic plug 194, and an electrosurgical plug 196. Ultrasonic plug 194 is configured for connection with ultrasonic plug port 230 of surgical generator 200 while electrosurgical plug 196 is configured for connection with bipolar electrosurgical plug port 240 of surgical generator 200 and/or active monopolar electrosurgical plug port 250 of surgical generator 200. In configurations where generator 200 includes a common port, cable assembly 190 may include a common plug (not shown) configured to act as both the ultrasonic plug 194 and the electrosurgical plug 196.

Plural first electrical lead wires 197 electrically coupled to ultrasonic plug 194 extend through cable 192 and into handle assembly 110 for electrical connection to ultrasonic transducer 140 and/or activation button 120 to enable the selective supply of ultrasonic drive signals from surgical generator 200 to ultrasonic transducer 140 upon activation of ultrasonic energy. In addition, plural second electrical lead wires 199 are electrically coupled to electrosurgical plug 196 and extend through cable 192 into handle assembly 110. In bipolar configurations, separate second electrical lead wires 199 are electrically coupled to waveguide 154 and jaw member 164 (and/or different portions of jaw member 164) such that bipolar electrosurgical energy may be conducted between blade 162 and jaw member 164 (and/or between different portions of jaw member 164). In monopolar configurations, a second electrical lead wire 199 is electrically coupled to waveguide 154 such that monopolar electrosurgical energy may be supplied to tissue from blade 162. Alternatively or additionally, a second electrical lead wire 199 may electrically couple to jaw member 164 in the monopolar configuration to enable monopolar electrosurgical energy to be supplied to tissue from jaw member 164. In configurations where both bipolar and monopolar functionality are enabled, one or more of the second electrical lead wires 199 may be used for both the delivery of bipolar energy and monopolar energy; alternatively, bipolar and monopolar energy delivery may be provided by separate second electrical lead wires 199. One or more other second electrical lead wires 199 is electrically coupled to activation button 120 to enable the selective supply of electrosurgical energy from surgical generator 200 to waveguide 154 and/or jaw member 164 upon activation of electrosurgical energy.

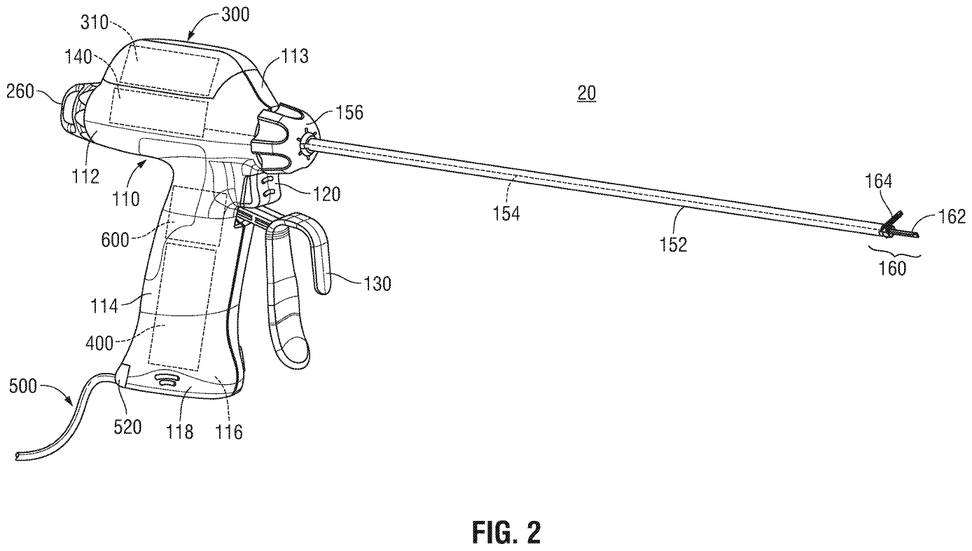

As an alternative to a remote generator 200, surgical system 10 may be at least partially cordless in that it incorporates an ultrasonic generator, an electrosurgical generator, and/or a power source, e.g., a battery, thereon or therein. In this manner, the connections from surgical instrument 100 to external devices, e.g., generator(s) and/or power source(s), is reduced or eliminated. More specifically, with reference to FIG. 2, another surgical system in accordance with the present disclosure is shown illustrated as a surgical instrument 20 supporting an ultrasonic generator 310, a power source (e.g., battery assembly 400), and an electrosurgical generator 600 thereon or therein. Surgical instrument 20 is similar to surgical instrument 100 (FIG. 1) and may include any of the features thereof except as explicitly contradicted below. Accordingly, only differences between surgical instrument 20 and surgical instrument 100 (FIG. 1) are described in detail below while similarities are omitted or summarily described.

Housing 112 of surgical instrument 20 includes a body portion 113 and a fixed handle portion 114 depending from body portion 113. Body portion 113 of housing 112 is configured to support an ultrasonic transducer and generator assembly (“TAG”) 300 including ultrasonic generator 310 and ultrasonic transducer 140. TAG 300 may be permanently engaged with body portion 113 of housing 112 or removable therefrom.

Fixed handle portion 114 of housing 112 defines a compartment 116 configured to receive battery assembly 400 and electrosurgical generator 600 and a door 118 configured to enclose compartment 116. An electrical connection assembly (not shown) is disposed within housing 112 and serves to electrically couple activation button 120, ultrasonic generator 310 of TAG 300, and battery assembly 400 with one another when TAG 300 is supported on or in body portion 113 of housing 112 and battery assembly 400 is disposed within compartment 116 of fixed handle portion 114 of housing 112, thus enabling activation of surgical instrument 20 in an ultrasonic mode in response to appropriate actuation of activation button 120. Further, the electrical connection assembly or a different electrical connection assembly disposed within housing 112 serves to electrically couple activation button 120, electrosurgical generator 600, battery assembly 400, and end effector assembly 160 (e.g., blade 162 and jaw member 164 and/or different portions of jaw member 164) with one another when electrosurgical generator 600 and battery assembly 400 are disposed within compartment 116 of fixed handle portion 114 of housing 112, thus enabling activation of surgical instrument 20 to supply electrosurgical energy, e.g., bipolar RF energy, in response to appropriate actuation of activation button 120. To enable the supply of monopolar electrosurgical energy, plug 520 of return electrode device 500 may be configured to connect to surgical instrument 20 (electrosurgical generator 600 thereof, more specifically), to complete a monopolar circuit through tissue and between surgical instrument 20 (e.g., blade 162 and/or jaw member 164) and return electrode device 500.

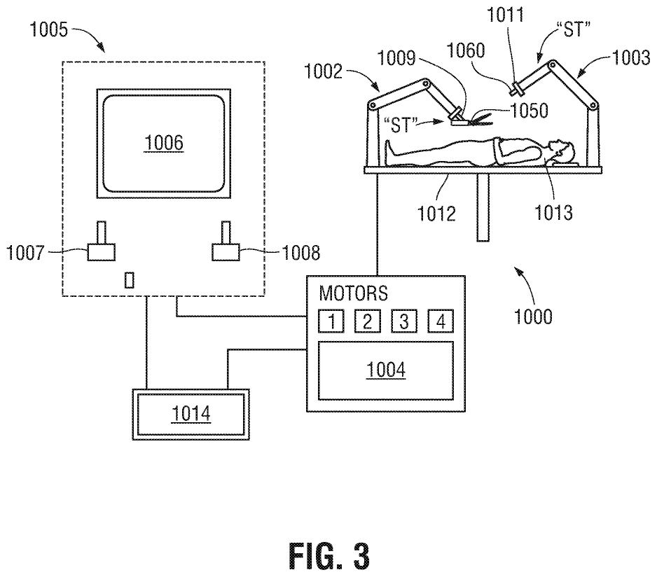

Turning to FIG. 3, a robotic surgical system in accordance with the aspects and features of the present disclosure is shown generally identified by reference numeral 1000. For the purposes herein, robotic surgical system 1000 is generally described. Aspects and features of robotic surgical system 1000 not germane to the understanding of the present disclosure are omitted to avoid obscuring the aspects and features of the present disclosure in unnecessary detail.

Robotic surgical system 1000 generally includes a plurality of robot arms 1002, 1003; a control device 1004; and an operating console 1005 coupled with control device 1004. Operating console 1005 may include a display device 1006, which may be set up in particular to display three dimensional images; and manual input devices 1007, 1008, by means of which a person (not shown), for example a surgeon, may be able to telemanipulate robot arms 1002, 1003 in a first operating mode. Robotic surgical system 1000 may be configured for use on a patient 1013 lying on a patient table 1012 to be treated in a minimally invasive manner. Robotic surgical system 1000 may further include a database 1014, in particular coupled to control device 1004, in which are stored, for example, pre-operative data from patient 1013 and/or anatomical atlases.

Each of the robot arms 1002, 1003 may include a plurality of members, which are connected through joints, and an attaching device 1009, 1011, to which may be attached, for example, a surgical tool “ST” supporting an end effector 1050, 1060. One of the surgical tools “ST” may be surgical instrument 100 (FIG. 1), surgical instrument 20 (FIG. 2), or any other suitable surgical instrument 20 configured for use in both an ultrasonic mode and one or more electrosurgical (bipolar and/or monopolar) modes, wherein manual actuation features, e.g., actuation button 120 (FIG. 1), clamp lever 130 (FIG. 1), etc., are replaced with robotic inputs. Robotic surgical system 1000 may further include or be configured to connect to an ultrasonic generator, an electrosurgical generator, and/or a power source. The other surgical tool “ST” may include any other suitable surgical instrument, e.g., an endoscopic camera, other surgical tool, etc. Robot arms 1002, 1003 may be driven by electric drives, e.g., motors, that are connected to control device 1004. Control device 1004 (e.g., a computer) may be configured to activate the motors, in particular by means of a computer program, in such a way that robot arms 1002, 1003, their attaching devices 1009, 1011, and, thus, the surgical tools “ST” execute a desired movement and/or function according to a corresponding input from manual input devices 1007, 1008, respectively. Control device 1004 may also be configured in such a way that it regulates the movement of robot arms 1002, 1003 and/or of the motors.

Referring to FIGS. 4-6, end effector assembly 160 of surgical instrument 100 of surgical system 10 (FIG. 1) is detailed, although the aspects and features of end effector assembly 160 may similarly apply, to the extent consistent, to surgical instrument 20 (FIG. 2) and/or any other suitable surgical instrument or system. End effector assembly 160, as noted above, includes blade 162 and jaw member 164. Blade 162 may define a linear configuration, may define a curved configuration, or may define any other suitable configuration, e.g., straight and/or curved surfaces, portions, and/or sections; one or more convex and/or concave surfaces, portions, and/or sections; etc. With respect to curved configurations, blade 162, more specifically, may be curved in any direction relative to jaw member 164, for example, such that the distal tip of blade 162 is curved towards jaw member 164, away from jaw member 164, or laterally (in either direction) relative to jaw member 164. Further, blade 162 may be formed to include multiple curves in similar directions, multiple curves in different directions within a single plane, and/or multiple curves in different directions in different planes. In addition, blade 162 may additionally or alternatively be formed to include any suitable features, e.g., a tapered configuration, various different cross-sectional configurations along its length, cut outs, indents, edges, protrusions, straight surfaces, curved surfaces, angled surfaces, wide edges, narrow edges, and/or other features.

Blade 162 may define a polygonal, rounded polygonal, or any other suitable cross-sectional configuration(s). Waveguide 154 or at least the portion of waveguide 154 proximally adjacent blade 162, may define a cylindrical shaped configuration. Plural tapered surfaces (not shown) may interconnect the cylindrically shaped waveguide 154 with the polygonal (rounded edge polygonal, or other suitable shape) configuration of blade 162 to define smooth transitions between the body of waveguide 154 and blade 162.

Blade 162 may be wholly or selectively coated with a suitable material, e.g., a non-stick material, an electrically insulative material, an electrically conductive material, combinations thereof, etc. Suitable coatings and/or methods of applying coatings include but are not limited to Teflon®, polyphenylene oxide (PPO), deposited liquid ceramic insulative coatings; thermally sprayed coatings, e.g., thermally sprayed ceramic; Plasma Electrolytic Oxidation (PEO) coatings; anodization coatings; sputtered coatings, e.g., silica; ElectroBond® coating available from Surface Solutions Group of Chicago, IL, USA; or other suitable coatings and/or methods of applying coatings.

Continuing with reference to FIGS. 4-6, blade 162, as noted above, in addition to receiving ultrasonic energy transmitted along waveguide 154 from ultrasonic transducer 140 (FIG. 1), is adapted to connect to generator 200 (FIG. 1) to enable the supply of RF energy to blade 162 for conduction to tissue in contact therewith. In bipolar configurations, RF energy is conducted between blade 162 and jaw member 164 (or between portions of jaw member 164 and/or blade 162) and through tissue disposed therebetween to treat tissue. In monopolar configurations, RF energy is conducted from blade 162, serving as the active electrode, to tissue in contact therewith and is ultimately returned to generator 200 (FIG. 1) via return electrode device 500 (FIG. 1), serving as the passive or return electrode.

Jaw member 164 of end effector assembly 160 includes more rigid structural body 182 and more compliant jaw liner 184. Structural body 182 may be formed from an electrically conductive material, e.g., stainless steel, and/or may include electrically conductive portions. Structural body 182 includes a pair of proximal flanges 183a that are pivotably coupled to the inner support sleeve 153 via receipt of pivot bosses (not shown) of proximal flanges 183a within corresponding openings (not shown) defined within the inner support sleeve 153 and operably coupled with outer drive sleeve 152 via a drive pin 155 secured relative to outer drive sleeve 152 and pivotably received within apertures 183b defined within proximal flanges 183a. As such, sliding of outer drive sleeve 152 about inner support sleeve 153 pivots jaw member 164 relative to blade 162 from the open position towards the closed position to clamp tissue between jaw liner 184 of jaw member 164 and blade 162.

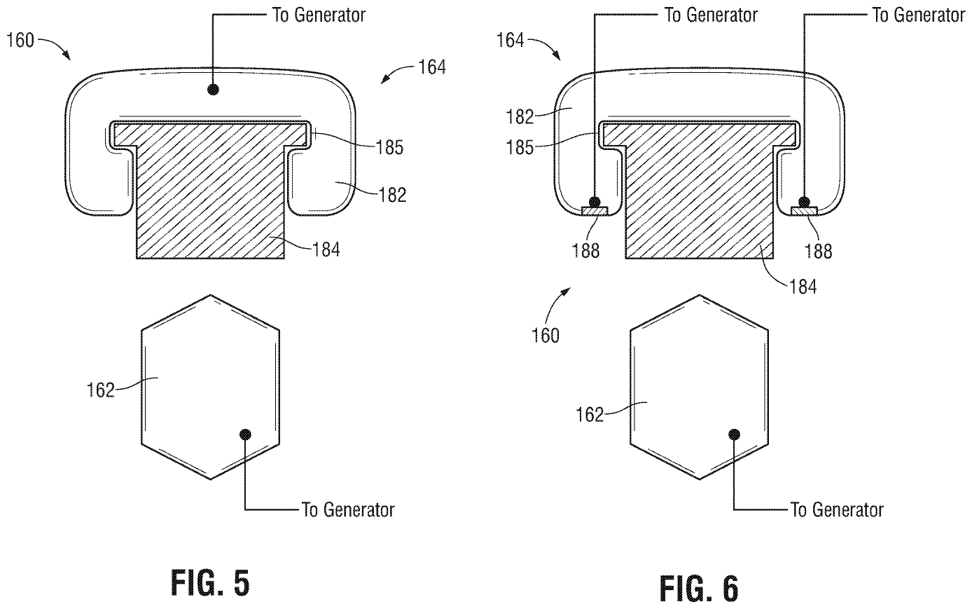

With reference to FIG. 5, structural body 182 may be adapted to connect to a source of electrosurgical energy, e.g., generator 200 (FIG. 1), and, in a bipolar configuration, is charged to a different potential as compared to blade 162 to enable the conduction of bipolar electrosurgical (e.g., RF) energy through tissue clamped therebetween, to treat the tissue. In a monopolar configuration, structural body 182 may be un-energized, may be charged to the same potential as compared to blade 162 (thus both defining the active electrode), or may be energized while blade 162 is not energized (wherein structural body 182 defines the active electrode). In either monopolar configuration, energy is returned to generator 200 (FIG. 1) via return electrode device 500 (FIG. 1), which serves as the passive or return electrode.

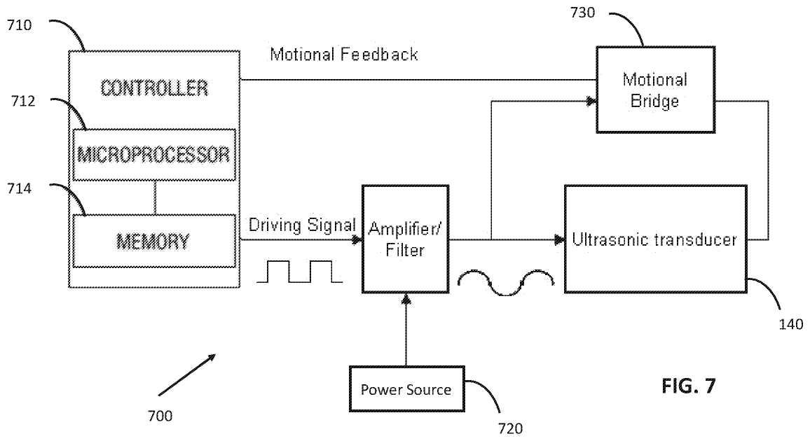

Referring to FIG. 6, as an alternative to the entirety of structural body 182 of jaw member 164 being connected to generator 200 (FIG. 1), the structural body may be formed from or embedded at least partially in an insulative material, e.g., an overmolded plastic. In such configurations, electrically conductive surfaces 188, e.g., in the form of plates, may be disposed on or captured by the overmolded plastic to define electrodes on either side of jaw liner 184 on the blade facing side of jaw member 164. The electrically conductive surfaces 188, in such aspects, are connected to generator 200 (FIG. 1) and may be energized for use in bipolar and/or monopolar configurations, e.g., energized to the same potential as one another and/or blade 162 and/or different potentials as one another and/or blade 162. In aspects, electrically conductive surfaces 188 are disposed at additional or alternative locations on jaw member 164, e.g., along either or both sides thereof, along a back surface thereof, etc.

Returning to FIGS. 4-6, jaw liner 184 is shaped complementary to a cavity 185 defined within structural body 182, e.g., defining a T-shaped configuration, to facilitate receipt and retention therein, although other configurations are also contemplated. Jaw liner 184 is fabricated from an electrically insulative, compliant material such as, for example, polytetrafluoroethylene (PTFE). The compliance of jaw liner 184 enables blade 162 to vibrate while in contact with jaw liner 184 without damaging components of ultrasonic surgical instrument 100 (FIG. 1) and without compromising the hold on tissue clamped between jaw member 164 and blade 162. Jaw liner 184 extends from structural body 182 towards blade 162 to inhibit contact between structural body 182 and blade 162 in the closed position of jaw member 164. The insulation of jaw liner 184 maintains electrical isolation between blade 162 and structural body 182 of jaw member 164, thereby inhibiting shorting.

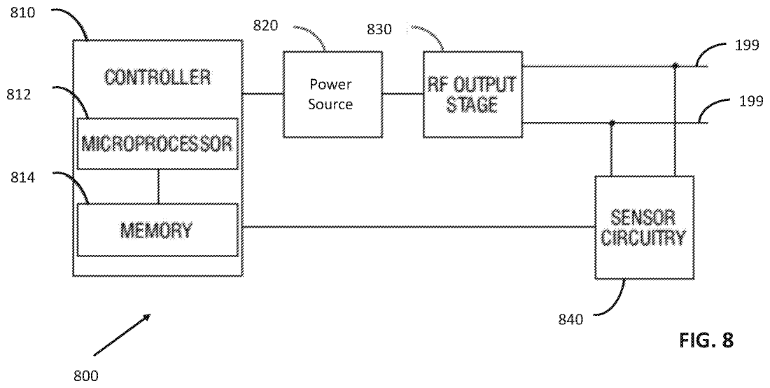

Turning to FIG. 7, an ultrasonic control system 700 of surgical instrument 100 of surgical system 10 (see FIG. 1) is detailed, although the aspects and features of ultrasonic control system 700 may similarly apply, to the extent consistent, to surgical instrument 20 (FIG. 2) and/or any other suitable surgical instrument or system. Ultrasonic control system 700 includes a controller 710 (e.g., of generator 200 (FIG. 1), ultrasonic generator 310 (FIG. 2), or any other suitable generator) and a power source 720 (e.g., generator 200 (FIG. 1), battery 400 (FIG. 2), or any other suitable power source) configured to control and power the electrical input to the ultrasonic transducer 140. Controller 710, more specifically, includes a microprocessor 712 and memory 714, e.g., storing instructions to be executed by microprocessor 712 to control the ultrasonic drive signal provided to ultrasonic transducer 140.

Ultrasonic control system 700 further includes a motional bridge 730 configured to sense a mechanical motion, e.g., a magnitude and frequency of mechanical motion, of ultrasonic transducer 140. The mechanical motion feedback provided by motional bridge 730 to controller 710 enables the controller 710 to control the frequency and/or magnitude of the driving signal, e.g., the high voltage AC driving signal, provided to ultrasonic transducer 140 to achieve a target amount of mechanical motion of ultrasonic transducer 140 at its resonance frequency and, thus, in aspects, a target blade velocity of blade 162 (FIGS. 4-6). Controller 710 is also configured to monitor the resonant frequency of ultrasonic transducer 140, which varies throughout use such as, for example, due to changes in load applied to blade 162 (FIG. 4), temperature of blade 162 (FIG. 4), and/or other factors.

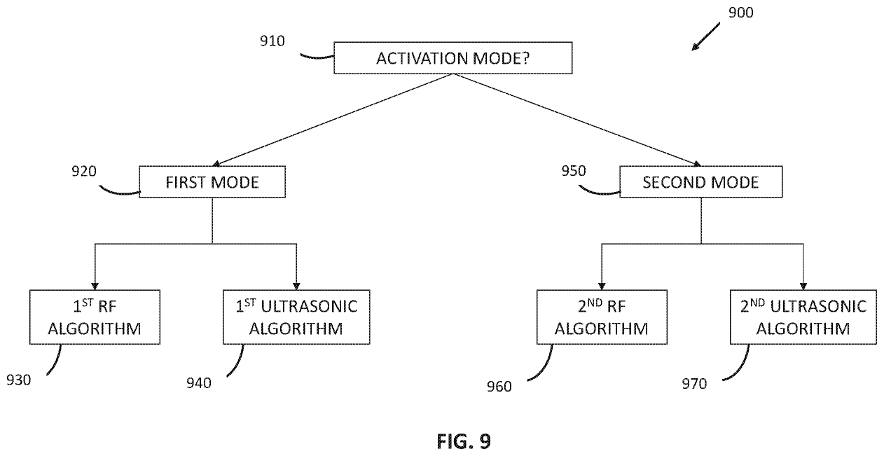

Referring to FIG. 8, an electrosurgical control system 800 of surgical instrument 100 of surgical system 10 (see FIG. 1) is detailed, although the aspects and features of electrosurgical control system 800 may similarly apply, to the extent consistent, to surgical instrument 20 (FIG. 2) and/or any other suitable surgical instrument or system. Electrosurgical control system 800 includes a controller 810 (e.g., of generator 200 (FIG. 1), electrosurgical generator 600 (FIG. 2), or any other suitable generator), a power source 820 (e.g., generator 200 (FIG. 1), battery 400 (FIG. 2), or any other suitable power source), an RF output stage 830, and sensor circuitry 840. Controller 810 includes a microprocessor 812 and memory 814, e.g., storing instructions to be executed by microprocessor 812 to control the electrosurgical energy output by RF output stage 830. Controller 810 (and/or microprocessor 812 and/or memory 814) may be the same controller 710 (and/or microprocessor 712 and/or memory 714) utilized in the ultrasonic control system 700 (FIG. 7) or may be a separate controller 810 (and/or microprocessor 812 and/or memory 814). Likewise, power source 820 may be the same power source 720 utilized in the ultrasonic control system 700 (FIG. 7) or may be a separate power source 820.

With additional reference to FIG. 4, RF output stage 830, in a bipolar configuration(s), is configured to supply electrosurgical energy to jaw member 164, a portion of jaw member 164, and/or blade 162 of end effector assembly 160 via one of electrical lead wires 199 and to return energy from jaw member 164, a portion of jaw member 164, and/or blade 162 of end effector assembly 160 to complete the circuit back to electrosurgical control system 800 via the other electrical lead wire 199. In a monopolar configuration(s), RF output stage 830 supplies electrosurgical energy to jaw member 164, a portion of jaw member 164, and/or blade 162 of end effector assembly 160 via one of electrical lead wires 199 to treat tissue while the energy is returned to complete the circuit back to electrosurgical control system 800 via return device 500 (FIG. 1).

Sensor circuitry 840 is operably coupled to wires 199 (or one wire 199 and return device 500 (FIG. 1)) so as to sense electrical parameters of the energy delivered to end effector assembly 160, e.g., voltage, current, resistance, etc. thereof, and, based thereon, determine one or more parameters of tissue, e.g., impedance of tissue, which can be utilized to determine whether tissue is sufficiently sealed (or a stage of tissue in the tissue sealing process), transected, or otherwise treated. Sensor circuitry 840 provides feedback, e.g., based on the sensed electrical parameter(s), to controller 810 and/or controller 710 of electrosurgical control system 800 and/or ultrasonic control system 700 (FIG. 7), which, in turn, implement energy-delivery algorithms, select energy-delivery algorithms, modify energy-delivery algorithms, and/or adjust energy-delivery parameters based thereon.

In addition or as an alternative to electrosurgical control system 800 controlling the supply of electrosurgical energy to treat tissue, electrosurgical control system 800 may also be configured to supply energy to tissue to interrogate tissue, e.g., wherein sensor circuitry 840 senses one or more electrical parameters to provide feedback to controller 810 such as, for example, to enable determination of the impedance of tissue. That is, system 10 (FIG. 1), system 20 (FIG. 2), or any other suitable instrument or system in accordance with the present disclosure may be configured to treat tissue with ultrasonic energy while interrogating tissue with electrosurgical energy (during, prior to, after, intermittently of, or without supplying tissue-treating electrosurgical energy). Tissue interrogation may be initiated in a bipolar electrosurgical interrogation mode and/or a monopolar electrosurgical interrogation mode. In aspects, electrosurgical interrogating may be performed in the absence of any treatment energy, e.g., with the ultrasonic energy turned off, e.g., to assess tissue before treatment (e.g., to determine a type of treatment, a suitable energy delivery algorithm, and/or suitable energy delivery parameter), after treatment (e.g., to determine completion of tissue treatment and/or a state of treated or surrounding tissue), or in other circumstances. Tissue interrogation may also be accomplished in conjunction with the supply of tissue-treating electrosurgical energy (e.g., simultaneously with or otherwise in conjunction with the supply of tissue-treating electrosurgical energy).

With respect to interrogation of tissue, electrosurgical control system 800 is configured to transmit an interrogation signal according to any of the electrosurgical paths detailed above such that the signal is returned to electrosurgical control system 800 to enable evaluation thereof. The interrogation signal may be a continuous signal, a pulse signal, or a plurality of pulses. Electrosurgical control system 800, more specifically, is configured to evaluate the returned signal, e.g., the voltage, current, resistance, etc. thereof, and, based thereon, determine one or more parameters of tissue, e.g., the impedance of tissue, which is indicative of whether tissue is sufficiently sealed. Electrosurgical control system 800 and/or ultrasonic control system 700 (FIG. 7), in turn, implement energy-delivery algorithms, select energy-delivery algorithms, modify energy-delivery algorithms, and/or adjust energy-delivery parameters based thereon.

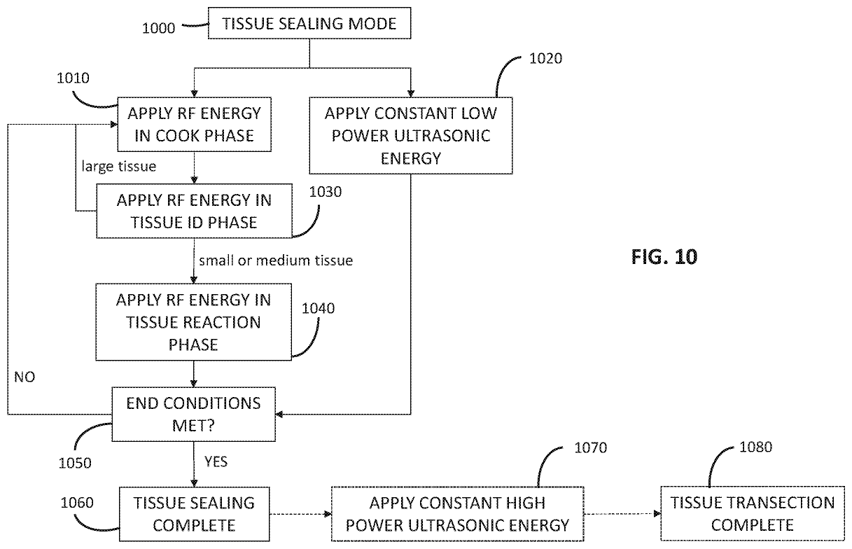

Turning to FIG. 9, electrosurgical energy and ultrasonic energy may be supplied simultaneously, consecutively, intermittently, combinations thereof, or in any other suitable manner according to one or more tissue treatment modes. For example, the instruments and systems of the present disclosure may be configured to operate in a first activation mode, e.g., a tissue sealing mode, and a second activation mode, e.g., a tissue transection mode, and to apply electrosurgical and/or ultrasonic energy differently based upon the operational mode selected. As noted above, mode selection may be provided by activation button 120 (FIG. 1) or any other suitable user-input. Alternatively or additionally, mode selection may be determined automatically based on sensed feedback regarding tissue, the instrument or system, or in any other suitable manner.

Continuing with reference to FIG. 9, method 900 of treating tissue, e.g., tissue clamped between jaw member 164 and blade 162 (FIGS. 1 and 4), in accordance with the present disclosure begins at 910 where the activation mode is determined, e.g., based upon a signal received from activation button 120 (FIG. 1), sensed feedback, and/or in any other suitable manner. Where a first activation mode is determined, as indicated at 920, e.g., based upon actuation of activation button 120 (FIG. 1) to a first activated state, first electrosurgical (RF) and ultrasonic algorithms are implemented to treat tissue, as indicated at 930 and 940, respectively, in accordance with the first activation mode. Where the second activation mode is determined, as indicated at 950, e.g., based upon actuation of activation button 120 (FIG. 1) to a second activated state, second electrosurgical (RF) and ultrasonic algorithms are implemented to treat tissue, as indicated at 960 and 970, respectively. Electrosurgical (RF) and ultrasonic energy may be delivered simultaneously in one or both of the first and second activation modes, although other configurations are also contemplated for either or both modes such as, for example, alternative energy delivery, sequential energy delivery, offset and overlapping energy delivery, delivery of only one energy modality, etc.

The first and second electrosurgical (RF) algorithms may be different from one another and/or the first and second ultrasonic algorithms may be different from one another. That is, the first and second activation modes made be different from one another at least with respect to the application of electrosurgical energy and/or with respect to the application of ultrasonic energy.

The electrosurgical (RF) algorithms may differ from one another, for example, with respect to parameter(s) controlled, profile, combinations thereof, etc. With respect to parameter(s) controlled, the first and second electrosurgical (RF) algorithms (or portions thereof) may correspond to the application of electrosurgical (RF) energy utilizing different parameter-based control such as, for example: voltage control, current control, impedance control, power control, combinations thereof, etc.

With respect to profile, the first and second electrosurgical (RF) algorithms (or portions thereof) may correspond to the application of electrosurgical (RF) energy utilizing different energy profiles by implementing different functions, combinations of functions, and/or durations of functions such as, for example: constant functions, stepped functions, ramped functions, polynomial functions, etc., wherein the functions may be based on the same or different parameters (e.g., voltage, current, power, impedance, etc.). Additionally or alternatively, the energy profile functions may differ, for example, as continuous energy application (constant or varied) versus pulsed energy application (and/or different pulsed patterns, durations, etc.).

The ultrasonic algorithms may differ from one another, for example, with respect to power level, profile, combinations thereof, etc. With respect to power level, the amplitude of the drive signal may be controlled to achieve a target velocity of blade motion by controlling the current supplied to the ultrasonic transducer 140 (FIG. 7) based upon the motional current feedback signal, which is an analog of blade velocity. Alternatively, the power level may correspond to the current or voltage input to the ultrasonic transducer 140 (FIG. 7) or may be determined in any other suitable manner. In aspects, for example, the first ultrasonic algorithm (or a portion thereof) may correspond to a first power level, wherein blade 162 (FIGS. 1 and 4) is driven to vibrate at one or more first velocities, while the second ultrasonic algorithm (or a portion thereof) corresponds to a second, different power level, wherein blade 162 (FIGS. 1 and 4) is driven to vibrate at one or more second, different velocities. In such configurations, the first and second velocities may be constants, e.g., wherein the blade 162 (FIGS. 1 and 4) is driven to vibrate at a constant first velocity in the first ultrasonic algorithm (or a portion thereof) and at a constant second velocity (different from the constant first velocity) in the second ultrasonic algorithm (or a portion thereof). In other configurations, the first and second power levels (e.g., blade velocities) may be variable over time and may define different set points, ramps, durations, etc. such that the power levels are different for at least portions of the first and second ultrasonic algorithms.

With respect to profile, the first and second ultrasonic algorithms (or portions thereof) may correspond to the application of different ultrasonic energy profiles by implementing different functions, combinations of functions, and/or durations of functions such as, for example: constant functions, stepped functions, ramped functions, polynomial functions, etc., wherein the functions may be output functions (e.g., blade velocity) or input functions (e.g., voltage or current of the drive signal).

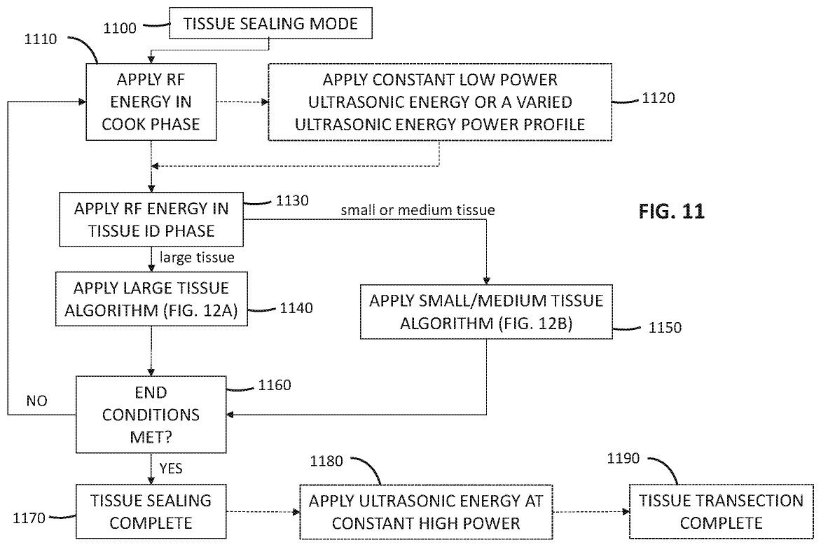

In aspects, as noted above, the first activation mode may correspond to a tissue sealing mode while the second activation mode corresponds to a tissue transection mode. Various configurations of the tissue sealing modes and aspects and features thereof are described below with reference to FIGS. 10-14. With respect to the tissue transection mode, RF energy and ultrasonic energy may be supplied simultaneously. The RF energy may be supplied at constant voltage (e.g., according to a constant voltage algorithm). The ultrasonic energy may be supplied according to a constant power level function, e.g., according to a constant blade velocity algorithm whereby blade velocity is held constant. The power level or blade velocity may correspond to a relatively high blade velocity of, in aspects, from about 8 m/s to about 12 m/s, in other aspects, from about 9 m/s to about 11 m/s, and in still other aspects from about 10.0 m/s to about 10.5 m/s.

Turning to FIG. 10, a tissue sealing mode 1000 is described. Initially, e.g., upon activation in the tissue sealing mode, RF energy is applied in a cook phase 1010 and ultrasonic energy is simultaneously applied at a constant low power ultrasonic energy level 1020. In the cook phase of RF energy application 1010, RF energy application may be controlled according to a polynomial curve, such as of current, voltage, or power. In aspects, RF power may be within a range of power of from about 1 W to about 25 W and RF voltage may be within a range of from about 25 Vrms to about 50 Vrms. The constant low power ultrasonic energy level may correspond to a blade velocity of, in aspects, from about 3 m/s to about 8 m/s, in other aspects, from about 5 m/s to about 7 m/s, and in still other aspects from about 6.0 m/s to about 6.5 m/s.

Once the cook phase 1010 is complete, and while the constant low power ultrasonic energy 1020 is continually applied, the RF energy is utilized to identify the tissue being treated in a tissue ID phase 1030. The tissue may be identified in tissue ID phase 1030 as small/medium tissue or large tissue, for example, and may be identified based on end impedance after the cook phase 1010 is complete or in any other suitable manner. Where small or medium tissue is identified, and while the constant low power ultrasonic energy 1020 is continually applied, RF energy is applied in tissue reaction phase 1040. RF energy may be controlled, e.g., by controlling power, in the tissue reaction phase 1040 to achieve a target tissue impedance trajectory based on measured tissue impedance and a predetermined rise in tissue impedance (tissue impedance ramp). Where large tissue is identified, the process may return to repeat the cook phase before moving to the tissue reaction phase 1040.

At completion of the tissue reaction phase 1040, it is determined whether tissue sealing end conditions are met at 1050. The determination of whether tissue sealing end conditions are met at 1050 may be based on, for example, whether a target impedance threshold is reached. Sensor feedback may additionally or alternatively be used to determine whether end conditions are met, e.g., sensors configured to sense tissue temperature (that is compared to an end temperature condition), tissue color or opacity (that are compared to tissue color or opacity end conditions), tissue water content (that is compared to an end tissue water content condition), jaw aperture or jaw angle (that are compared to jaw aperture or jaw angle end conditions), etc. If tissue sealing end conditions are not met at 1050, the process returns to repeat the tissue sealing mode 1000 or portions thereof. Alternatively, where sealing end conditions are not met at 1050, an error may be returned wherein an indicator (e.g., visual indicator, audible tone, combinations thereof, etc.) is output from the generator, instrument, and/or system and/or where all energy delivery is terminated.

If tissue sealing end conditions are met at 1050, both RF and ultrasonic energy delivery are terminated, tissue sealing is determined to be complete as indicated at 1060, and a corresponding indicator (e.g., visual indicator, audible tone, combinations thereof, etc.) is provided from the generator, instrument, and/or system to indicate completion of the tissue seal.

Continuing with reference to FIG. 10, in aspects, once tissue sealing is complete as indicated at 1060, the sealed tissue may be transected. In particular, upon application of user input (from a button, switch, or other suitable input) to transect tissue, e.g., by switching to the second mode of operation after tissue sealing or automatically based upon the first mode of operation being a tissue sealing and transection mode, the process proceeds to 1070 where ultrasonic energy is applied (while RF energy remains OFF) at the constant high power ultrasonic energy level 1020. As noted above, the power level or blade velocity at high power may correspond to a relatively high blade velocity of, in aspects, from about 8 m/s to about 12 m/s, in other aspects, from about 9 m/s to about 11 m/s, and in still other aspects from about 10.0 m/s to about 10.5 m/s. Tissue transection is completed at 1080 after a pre-determined duration of application of constant high power ultrasonic energy, in accordance with sensed feedback, or in any other suitable manner.

Referring to FIG. 11, another tissue sealing mode 1100 is described. Features of tissue sealing mode 1100 similar to those of tissue sealing mode 1000 (FIG. 10) are summarily described or omitted entirely for purposes of brevity. Initially, e.g., upon activation in the tissue sealing mode, RF energy is applied in a cook phase 1110. In aspects, ultrasonic energy is simultaneously applied at the constant low power ultrasonic energy level or according to a varied ultrasonic energy power profile, as indicated at 1120. With respect to the varied ultrasonic energy power profile, ultrasonic energy may be delivered to initially achieve a first blade velocity for a first time and to subsequently achieve a second blade velocity for a second time. The transition between the first and second blade velocities may be a step or a ramp (e.g., wherein the velocity is varied over time at a defined rate or rates). In aspects, the first blade velocity corresponds to the high power level and the second blade velocity corresponds to the low power level. Further, in aspects, the first time is a relatively shorter time, e.g., less than 3 seconds, less than 2 second, or less than 1 second, while the second time is a relatively longer time, e.g., greater than 3 seconds, greater than 4 seconds, or greater than 5 seconds. In other aspects, no ultrasonic energy is simultaneously applied during the RF cook phase 1110.

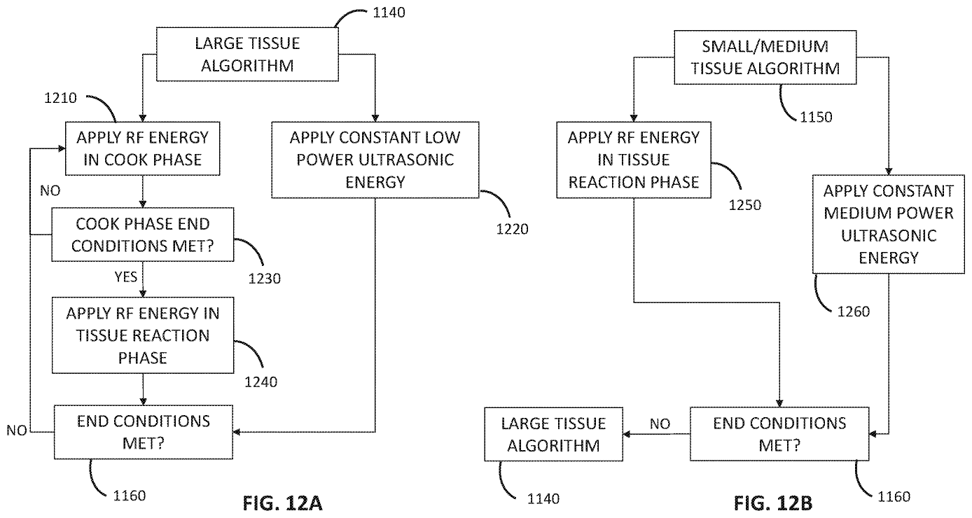

Once the cook phase 1110 is complete, RF energy is utilized to identify the tissue being treated in a tissue ID phase 1130. The tissue may be identified in tissue ID phase 1130 as small/medium tissue or large tissue, for example, and may be identified based on end impedance after the cook phase 1110 is complete or in any other suitable manner. Where large tissue is identified, a large tissue algorithm 1140 is implemented. On the other hand, where small/medium tissue is identified, a small/medium tissue algorithm 1150 is implemented. The large tissue algorithm 1140 and the small/medium tissue algorithm 1150 are described below with reference to FIGS. 12A and 12B, respectively.

After the large tissue algorithm 1140 or the small/medium tissue algorithm 1150 is completed, it is determined whether tissue sealing end conditions are met at 1160. If yes, tissue sealing is complete, as indicated at 1170 and, if tissue transection is desired, high power ultrasonic energy is provided at 1180 to transect tissue to complete tissue transection, as indicated at 1190. If the tissue sealing end conditions are not met, the process returns to cook phase 1110 or an error is returned.

With reference to FIG. 12A, with respect to the large tissue algorithm 1140, RF energy is applied in a cook phase 1210 and ultrasonic energy is simultaneously applied at the constant low power ultrasonic energy level 1220. At the end of the cook phase 1210, it is determined whether cook phase end conditions are met, as indicated at 1230. If the conditions are not met, the large tissue algorithm 1140 is re-started or an error is returned. If the conditions are met, and while the constant low power ultrasonic energy 1220 is continually applied, RF energy is applied in a tissue reaction phase 1240. At completion of the tissue reaction phase 1240, the process proceeds to 1160 where it is determined whether tissue sealing end conditions are met. The process proceeds from 1160 as detailed above (see FIG. 11).

Referring to FIG. 12B, with respect to the small/medium tissue algorithm 1150, RF energy is applied in a tissue reaction phase 1250 and ultrasonic energy is simultaneously applied at a constant medium power ultrasonic energy level 1260. The power level or blade velocity at medium power may correspond to a medium blade velocity of, in aspects, from about 6 m/s to about 10 m/s, in other aspects, from about 7 m/s to about 9 m/s, and in still other aspects from about 7.5 m/s to about 8.5 m/s.

At completion of tissue reaction phase 1250, the process proceeds to 1160 where it is determined whether tissue sealing end conditions are met. If, after the small/medium tissue algorithm 1150, tissue sealing end conditions are determined not to be met at 1160, the large tissue algorithm 1140 may be implemented, as detailed above, or an error indicator may be provided. If the tissue sealing end conditions are met, the process proceeds from 1160 as detailed above (see FIG. 11).

In alternate or additional aspects, the electrosurgical energy delivery algorithm in the tissue sealing mode may be any of the tissue sealing algorithms detailed in U.S. Pat. Nos.: 8,147,485; 8,685,016; 8,920,421; 9,186,200; and/or 10,617,463, the entire contents of each of which is hereby incorporated herein by reference.

While several aspects of the disclosure have been detailed above and are shown in the drawings, it is not intended that the disclosure be limited thereto, as it is intended that the disclosure be as broad in scope as the art will allow and that the specification be read likewise. Therefore, the above description and accompanying drawings should not be construed as limiting, but merely as exemplifications of particular aspects. Those skilled in the art will envision other modifications within the scope and spirit of the claims appended hereto.

What is claimed is:

1. A surgical system, comprising:
an electrosurgical system including first and second electrodes configured to conduct electrosurgical energy therebetween and through tissue to treat tissue; and
an ultrasonic system including an ultrasonic blade, the ultrasonic system configured to ultrasonically vibrate the ultrasonic blade to treat tissue in contact with the ultrasonic blade,
wherein, in a first mode of operation, the electrosurgical system is configured to conduct the electrosurgical energy according to a first electrosurgical algorithm and the ultrasonic system is configured to ultrasonically vibrate the ultrasonic blade according to a first ultrasonic algorithm,
wherein, in a second mode of operation, the electrosurgical system is configured to conduct the electrosurgical energy according to a second electrosurgical algorithm and the ultrasonic system is configured to ultrasonically vibrate the ultrasonic blade according to a second ultrasonic algorithm, and
wherein, at least one of: the first and second electrosurgical algorithms are different from one another, or the first and second ultrasonic algorithms are different from one another.

2. The surgical system according to claim 1, further comprising a jaw member movable relative to the ultrasonic blade for clamping tissue between the jaw member and the ultrasonic blade, wherein the jaw member includes one of the first or second electrodes, and wherein the ultrasonic blade includes the other of the first or second electrodes.

3. The surgical system according to claim 1, wherein, in at least one of the first or second modes of operation, the electrosurgical energy is conducted and the ultrasonic blade is ultrasonically vibrated simultaneously.

4. The surgical system according to claim 1, wherein the first mode of operation is a tissue sealing mode of operation and wherein the second mode of operation is a tissue transection mode of operation.

5. The surgical system according to claim 1, wherein the first electrosurgical algorithm includes a variable function and wherein the second electrosurgical algorithm includes a constant function.

6. The surgical system according to claim 5, wherein the variable function includes a polynomial function.

7. The surgical system according to claim 1, wherein the first electrosurgical algorithm includes power control based on impedance and wherein the second electrosurgical algorithm includes voltage control.

8. The surgical system according to claim 1, wherein the first electrosurgical algorithm includes a plurality of different stages and wherein the second electrosurgical algorithm includes a single stage.

9. The surgical system according to claim 1, wherein the first ultrasonic algorithm includes a constant function at a first power level and wherein the second ultrasonic algorithm includes a constant function at a second, different power level.

10. The surgical system according to claim 1, wherein the first ultrasonic algorithm includes a variable function and wherein the second ultrasonic algorithm includes a constant function.

11. A surgical method, comprising:
receiving an input to operate in a first mode of operation or a second mode of operation;
in response to receiving the input to operate in the first mode of operation:
conducting electrosurgical energy between first and second electrodes and through tissue according to a first electrosurgical algorithm; and
ultrasonically vibrating an ultrasonic blade in contact with tissue according to a first ultrasonic algorithm; and
in response to receiving the input to operate in the second mode of operation:
conducting electrosurgical energy between the first and second electrodes and through tissue according to a second electrosurgical algorithm; and
ultrasonically vibrating the ultrasonic blade in contact with tissue according to a second ultrasonic algorithm,
wherein, at least one of: the first and second electrosurgical algorithms are different from one another, or the first and second ultrasonic algorithms are different from one another.

12. The surgical method according to claim 11, wherein, in at least one of the first or second modes of operation, the conducting electrosurgical energy and the ultrasonically vibrating are performed simultaneously.

13. The surgical method according to claim 11, wherein, in each of the first and second modes of operation, the conducting electrosurgical energy and the ultrasonically vibrating are performed simultaneously.

14. The surgical method according to claim 11, wherein the first mode of operation is a tissue sealing mode of operation and wherein the second mode of operation is a tissue transection mode of operation.

15. The surgical method according to claim 11, wherein the conducting electrosurgical energy according to the first electrosurgical algorithm includes implementing a variable function and wherein the conducting electrosurgical energy according to the second electrosurgical algorithm includes implementing a constant function.

16. The surgical method according to claim 15, wherein the variable function includes a polynomial function.

17. The surgical method according to claim 11, wherein the conducting electrosurgical energy according to the first electrosurgical algorithm includes controlling power based on impedance and wherein the conducting electrosurgical energy according to the second electrosurgical algorithm includes implementing voltage control.

18. The surgical method according to claim 11, wherein the conducting electrosurgical energy according to the first electrosurgical algorithm includes implementing a plurality of electrosurgical energy delivery stages and wherein the conducting electrosurgical energy according to the second electrosurgical algorithm includes implementing a single electrosurgical energy delivery stage.

19. The surgical method according to claim 11, wherein the ultrasonically vibrating according to the first ultrasonic algorithm includes implementing a constant function at a first power level and wherein the ultrasonically vibrating according to the second ultrasonic algorithm includes implementing a constant function at a second, different power level.

20. The surgical method according to claim 11, wherein the ultrasonically vibrating according to the first ultrasonic algorithm includes implementing a variable function and wherein the ultrasonically vibrating according to the second ultrasonic algorithm includes implementing a constant function.
